(19) United States
(12) Patent Application Publication
(10) Pub. No.: US 2025/0303400 A1
(43) Pub. Date: Oct. 2, 2025

(54) CATALYST FOR ORGANIC SYNTHESIS USE, AND METHOD FOR PRODUCING ORGANIC COMPOUND

(71) Applicant: ORGANO CORPORATION, Tokyo (JP)

(72) Inventors: Ayumu KATO, Tokyo (JP); Shinji NAKAMURA, Tokyo (JP)

(73) Assignee: ORGANO CORPORATION, Tokyo (JP)

(21) Appl. No.: 18/868,949

(22) PCT Filed: Feb. 16, 2023
(86) PCT No.: PCT/JP2023/005486

§ 371 Date: Nov. 25, 2024

(30) Foreign Application Priority Data
May 27, 2022 (JP) 2022-087141

(51) Int. Cl. B01J 31/08; C07D 317/36

(52) U.S. Cl. CPC B01J 31/08; C07D 317/36; B01J 2231/34

(57) ABSTRACT

Provided are: a catalyst for organic synthesis use, which has a high catalytic activity, can be easily separated by solid/liquid separation from a reaction system, and enables highly efficient synthesis of an organic compound; and a method for producing an organic compound using the catalyst. This catalyst for organic synthesis use includes an anionic exchange resin that has a hydrohalic acid salt of a tertiary amino group as an ion exchange group. This method for producing an organic compound uses, as a catalyst, an anionic exchange resin that has a hydrohalic acid salt of a tertiary amino group as an ion exchange group.

### CROSS REFERENCE TO RELATED APPLICATION

This application is a National Stage of International Application No. PCT/JP2023/005486 filed on Feb. 16, 2023, which claims priority to Japanese Patent Application No. 2022-087141 filed on May 27, 2022, the contents of each of the above-identified applications are incorporated herein by reference in their entirety including the specifications, claims, and abstracts.

### TECHNICAL FIELD

The present invention relates to a catalyst for organic synthesis used for synthesizing an organic compound, and a method for producing an organic compound using the catalyst.

### BACKGROUND

For example, cyclic carbonate synthesis catalysts and cyclic carbonate production methods used for synthesizing cyclic carbonates from an alkylene oxide and carbon dioxide have long been known in the prior art.

Examples of heterogeneous catalysts for synthesizing cyclic carbonates include strong anion exchange resins having quaternary ammonium groups with a halide ion counter ion as the ion exchange groups (see Patent Document 1), strong anion exchange resins with a moisture content of not more than 0.5% by weight (see Patent Document 2), and anion exchange resins having tertiary amino groups or quaternary ammonium groups with a silicate counter anion as the ion exchange groups (see Patent Document 3).

Further, examples of known methods for synthesizing cyclic carbonates from an alkylene oxide and carbon dioxide include a method conducted in the presence of water using, as a heterogeneous catalyst, a strong anion exchange resin having quaternary ammonium groups with an iodide ion counter ion as the ion exchange groups (see Non-Patent Document 1), and a method using a hydroiodide salt of a tertiary amine as a homogenous catalyst (see Non-Patent Document 2).

However, in methods using a strong anion exchange resin having quaternary ammonium groups as the ion exchange groups, a problem may arise in that the catalytic activity is inadequate. Further, in methods using a homogenous catalyst, removing the catalyst from the reaction system may be problematic.

Furthermore, examples of known homogenous catalyst for synthesizing oxazolidinones include tertiary ammonium salts having a halide counter ion (see Non-Patent Document 3).

However, in methods using a homogenous catalyst, removing the catalyst from the reaction system may be problematic.

### CITATION LIST

### Patent Literature

Patent Document 1: U.S. Pat. No. 2,773,070
Patent Document 2: JP H03-120270 A
Patent Document 3: JP 3568226 B

### Non-Patent Literature

Non-Patent Document 1: ACS Sustainable Chem. Eng. 2020, 8, 7993-8003
Non-Patent Document 2: ACS Sustainable Chem. Eng. 2017, 5, 7295-7301
Non-Patent Document 3: Green Chem. 2011, 13, 2351-2353

### SUMMARY

### Technical Problem

Objects of the present invention are to provide a catalyst for organic synthesis that exhibits superior catalytic activity, can be easily separated from the reaction system by solid-liquid separation, and is capable of synthesizing organic compounds with high efficiency, and to also provide a method for producing organic compounds using the catalyst.

### Solution to Problem

The present invention provides a catalyst for organic synthesis that comprises an anion exchange resin having a hydrohalide salt of a tertiary amino group as an ion exchange group.

In the catalyst for organic synthesis, the hydrohalide salt is preferably a hydrobromide salt or a hydroiodide salt.

The catalyst for organic synthesis is preferably used for synthesis of a cyclic carbonate.

The present invention also provides a method for producing an organic compound that uses, as a catalyst, an anion exchange resin having a hydrohalide salt of a tertiary amino group as an ion exchange group.

In the method for producing an organic compound, the hydrohalide salt is preferably a hydrobromide salt or a hydroiodide salt.

In the method for producing an organic compound, the organic compound is preferably a cyclic carbonate.

### Advantageous Effects of Invention

The present invention is able to provide a catalyst for organic synthesis that exhibits superior catalytic activity, can be easily separated from the reaction system by solid-liquid separation, and is capable of synthesizing organic compounds with high efficiency, as well as providing a method for producing organic compounds that uses the catalyst.

### DESCRIPTION OF EMBODIMENTS

Embodiments of the present invention are described below. These embodiments are merely examples of implementing the present invention, and the present invention is not limited to these embodiments.

### <Catalyst for Organic Synthesis>

A catalyst for organic synthesis according to an embodiment of the present invention is a catalyst for synthesizing organic compounds, and for example, may be a catalyst for synthesizing a cyclic carbonate from an alkylene oxide and carbon dioxide, or a catalyst for synthesizing an organic compound such as an oxazolidinone from an aziridine and carbon dioxide. This catalyst is an anion exchange resin having a hydrohalide salt of a tertiary amino group as an ion exchange group, and may be used as a heterogeneous catalyst.

The inventors of the present invention discovered that, in a reaction for synthesizing an organic compound, a weak anion exchange resin having a hydrohalide salt of a tertiary amino group as an ion exchange group functioned as a catalyst of superior catalytic activity that could be easily separated from the reaction system by solid-liquid separation and was capable of synthesizing a cyclic carbonate with high efficiency. For example, in the reaction represented by the reaction formula shown below for synthesizing a cyclic carbonate from an alkylene oxide and carbon dioxide, the catalyst for organic synthesis according to an embodiment of the present invention functioned as a catalyst of superior catalytic activity that could be easily separated from the reaction system by solid-liquid separation and was capable of synthesizing a cyclic carbonate with high efficiency.

For example, when synthesizing a cyclic carbonate from an alkylene oxide and carbon dioxide, by using, as a catalyst, the salt of a weak anion exchange resin having tertiary amino groups as functional groups and a hydrohalide, a heterogeneous catalyst can be provided which enables a cyclic carbonate to be obtained with high efficiency, and can then be easily separated from the reaction system by a solid-liquid separation. By using this catalyst, a cyclic carbonate can be synthesized with higher efficiency than that achievable using catalysts described in known literature.

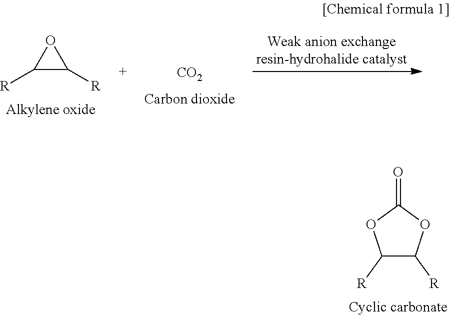

In the above reaction formula, there are no particular limitations on R in the alkylene oxide, provided the reaction is not inhibited, and for example, each R may independently represent a hydrogen atom; a linear, branched or cyclic alkyl group of 1 to 20 carbon atoms which may be substituted with a halogen atom, an aryl group of 6 to 15 carbon atoms, an alkoxy group of 1 to 8 carbon atoms, an oxo group, a nitro group, or a cyano group or the like; an alkenyl group of 2 to 12 carbon atoms; an alkynyl group of 2 to 12 carbon atoms; an aryl group of 6 to 18 carbon atoms; an alkoxy group of 1 to 8 carbon atoms; an acyl group of 1 to 8 carbon atoms; a halogen atom; a nitro group; or a cyano group or the like, although an alkyl group of 1 to 8 carbon atoms or an aryl group of 6 to 10 carbon atoms is preferred, and a methyl group or a phenyl group is more preferred.

There are no particular limitations on the alkylene oxide, provided the reaction is not inhibited, and examples include propylene oxide, ethylene oxide, 1,2-butylene oxide, propyloxirane, epichlorohydrin, styrene oxide, stilbene oxide, 4-chlorostyrene oxide, 1,2-epoxy-5-hexene, allyl glycidyl ether, and benzyl glycidyl ether. Preferred alkylene oxides include propylene oxide, epichlorohydrin, and styrene oxide.

Examples of the tertiary amino group of the catalyst for organic synthesis according to an embodiment of the present invention include tertiary amino groups such as a dimethylamino group, diethylamino group, dipropylamino group, dibutylamino group, methylhydroxyethylamino group, methylhydroxypropylamino group, dicyclohexylamino group, pyrrolidyl group, piperidyl group, 2,2,6,6-tetramethylpiperidyl group, and morpholyl group.

Examples of the halide ion that represents the counter ion include F, Cl, Br, and I, and in terms of safety and the like during preparation of the catalyst, Cl, Br or I is preferred, in terms of achieving a high reaction conversion rate and the like, Br or I is more preferred, and in terms of factors such as reducing by-products, I is particularly preferred.

There are no particular limitations on the type of polymer material that constitutes the skeleton of the anion exchange resin, and examples include crosslinked polymers including aromatic vinyl polymers such as polystyrene, poly(α-methylstyrene), polyvinyltoluene, poly(vinylbenzyl chloride), polyvinylbiphenyl, and polyvinylnaphthalene; polyolefins such as polyethylene and polypropylene; poly(halogenated polyolefins) such as polyvinyl chloride and polytetrafluoroethylene; nitrile-based polymers such as polyacrylonitrile; and (meth)acrylic-based polymers such as poly(methyl methacrylate), poly(glycidyl methacrylate), and poly(ethyl acrylate). These polymers may be polymers obtained by copolymerizing a single vinyl monomer and a crosslinking agent, polymers obtained by polymerizing a plurality of vinyl monomers and a crosslinking agent, or mixtures obtained by blending two or more different types of polymer. In terms of heat resistance and the like, the polymer material that constitutes the skeleton of the anion exchange resin is preferably a styrene-divinylbenzene copolymer resin.

The weak anion exchange resin having a hydrohalide salt of a tertiary amino group as an ion exchange group may be used, for example, in the form of a granular resin with a size of 300 to 1,000 μm.

### <Method for Producing Organic Compound>

A method for producing an organic compound according to an embodiment of the present invention is a method that uses, as a catalyst, an anion exchange resin having a hydrohalide salt of a tertiary amino group as an ion exchange group. For example, the method for producing an organic compound according to an embodiment of the present invention may be a method for synthesizing a cyclic carbonate from an alkylene oxide and carbon dioxide using, as a catalyst, an anion exchange resin having a hydrohalide salt of a tertiary amino group as an ion exchange group. Further, the method for producing an organic compound according to an embodiment of the present invention may also be, for example, a method for synthesizing an oxazolidinone from an aziridine and carbon dioxide using, as a catalyst, an anion exchange resin having a hydrohalide salt of a tertiary amino group as an ion exchange group.

In the reaction for synthesizing an organic compound using the catalyst for organic synthesis according to an embodiment of the present invention, there are no particular limitations on the reaction conditions, provided the conditions enable the target organic compound to be obtained. In the reaction for synthesizing a cyclic carbonate from an alkylene oxide and carbon dioxide using the catalyst for organic synthesis according to an embodiment of the present invention, there are no particular limitations on the reaction conditions, provided the conditions enable the target cyclic carbonate to be obtained. Further, in the reaction for synthesizing an oxazolidinone from an aziridine and carbon dioxide using the catalyst for organic synthesis according to an embodiment of the present invention, there are no particular limitations on the reaction conditions, provided the conditions enable the target oxazolidinone to be obtained.

In terms of the reaction conditions in the reaction for synthesizing a cyclic carbonate from an alkylene oxide and carbon dioxide using the catalyst for organic synthesis according to an embodiment of the present invention, the reaction temperature is, for example, within a range from 0 to 120° C., and preferably from 10 to 100° C. The reaction time is, for example, within a range from 1 to 96 hours, and preferably from 3 to 48 hours. The reaction pressure is, for example, within a range from atmospheric pressure to 5.0 MPa, and preferably from atmospheric pressure to 1.0 MPa. The reaction for synthesizing a cyclic carbonate using the catalyst for organic synthesis according to an embodiment of the present invention can also be conducted in a heterogeneous system, at normal temperature (15 to 30° C.) and atmospheric pressure.

There are no particular limitations on the solvent used in the reaction for synthesizing a cyclic carbonate from an alkylene oxide and carbon dioxide, provided the reaction is not impaired, and examples of the solvent include water; alcohol-based solvents such as methanol, ethanol, propanol, butanol, and benzyl alcohol; ketone-based solvents such as acetone, methyl ethyl ketone; nitrile-based solvents such as acetonitrile; amide-based solvents such as dimethylformamide, dimethylacetamide, and N-methylpyrrolidone; and ether-based solvents such as dimethyl ether and tetrahydrofuran, and one of these solvents may be used alone, or a mixture of two or more solvents may be used. In those cases where the alkylene oxide is in a liquid state under the reaction conditions, a solvent need not be used.

The reaction for synthesizing a cyclic carbonate from an alkylene oxide and carbon dioxide may be conducted, for example, by adding an alkylene oxide such as propylene oxide and a weak anion exchange resin having a hydrohalide salt of a tertiary amino group as an ion exchange group to a solvent such as water in a pressure-resistant reaction vessel, introducing carbon dioxide into the reaction vessel, and conducting a reaction heterogeneously at a prescribed temperature and a prescribed pressure for a prescribed period of time. Following reaction, the target cyclic carbonate can be obtained by removing the weak anion exchange resin using a solid-liquid separation technique such as filtration, and then removing the solvent by distillation or the like. In those cases where the alkylene oxide is in a liquid state under the reaction conditions, the reaction may be conducted, for example, by adding a weak anion exchange resin having a hydrohalide salt of a tertiary amino group as an ion exchange group to an alkylene oxide such as propylene oxide in a pressure-resistant reaction vessel, introducing carbon dioxide into the reaction vessel, and conducting a reaction heterogeneously at a prescribed temperature and a prescribed pressure for a prescribed period of time. Following reaction, the target cyclic carbonate can be obtained by removing the weak anion exchange resin using a solid-liquid separation technique such as filtration. The completion of the reaction can be confirmed, for example, by thin-layer chromatography (TLC), liquid chromatography (LC) measurement, gas chromatography (GC) measurement, nuclear magnetic resonance (NMR) measurement, or Fourier transform infrared spectroscopy (FT-IR) measurement or the like. The obtained cyclic carbonate may be purified using conventional methods.

By conducting this type of reaction, the cyclic carbonate can be obtained, for example, at a reaction conversion rate of at least 8%, and preferably 80% or higher.

### <Method for Producing Catalyst for Organic Synthesis>

The catalyst for organic synthesis according to an embodiment of the present invention is produced, for example, from an anion exchange resin having tertiary amino groups and a hydrogen halide.

In the reaction for synthesizing the catalyst for organic synthesis according to an embodiment of the present invention, there are no particular limitations on the reaction conditions, provided the conditions enable the anion exchange resin having a hydrohalide salt of the tertiary amino group as an ion exchange group to be obtained.

For example, the reaction may be conducted by mixing the anion exchange resin having tertiary amino groups and the hydrogen halide such as hydrogen chloride, hydrogen bromide or hydrogen iodide, and reacting the mixture at a prescribed temperature (for example, within a range from 10 to 30° C.) for a prescribed period of time (for example, within a range from 0.5 to 24 hours). The obtained resin may be washed with water or an alcohol-based solvent or the like.

Compared with the production process for a strong anion exchange resin having a quaternary ammonium group with a halide ion counter ion as an ion exchange group, the production process for a weak anion exchange resin having a hydrohalide salt of a tertiary amino group as an ion exchange group has fewer reaction steps such as the counter ion exchange step, and the weak anion exchange resin having a tertiary amino group as an ion exchange group can be easily converted to a hydrohalide salt.

### EXAMPLES

The present invention is described below in more specific detail using a series of examples and comparative examples, but the present invention is not limited to the following examples.

### Examples 1-1 to 1-3

Using, as a catalyst, an anion exchange resin having a hydrohalide salt of a tertiary amino group as an ion exchange group, and having the structure described below (namely, a salt of AmberLyst A21 (manufactured by Organo Corporation) and a hydrohalic acid), a synthesis reaction for 4-methyl-1,3-dioxolane-2-one shown below was conducted.

### (Reaction Conducted)

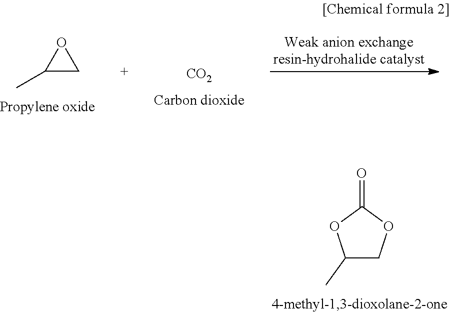

### (Chemical Structural Formula of Catalyst Used)

resin-N(CH₃)₂·HX
X═I (Examples 1-1 and 1-2), Br (Example 1-3)
resin=styrene-divinylbenzene copolymer resin

The synthesis reaction for the catalyst for organic synthesis (the cyclic carbonate synthesis catalyst) was conducted using the procedure described below.

### [Preparation of A21·HI]

In a reaction vessel, 2.17 g of a dried product of an anion exchange resin having tertiary amino groups (AmberLyst A21 (manufactured by Organo Corporation), resin-N(CH₃)₂), 30 mL of water, and 2.24 g of 55% to 58% hydroiodic acid were mixed together, and the mixture was reacted under stirring at room temperature (25° C.) for one hour. Subsequently, the reaction product was filtered, and the filtered residue was washed with water and then dried under reduced pressure, yielding the target catalyst for organic synthesis A21·HI.

### [Preparation of A21·HBr]

In a reaction vessel, 1.08 g of a dried product of an anion exchange resin having tertiary amino groups (AmberLyst A21 (manufactured by Organo Corporation)), 15 mL of water, and 0.827 g of 49% hydrobromic acid were mixed together, and the mixture was reacted under stirring at room temperature (25° C.) for one hour. Subsequently, the reaction product was filtered, and the filtered residue was washed with water and then dried under reduced pressure, yielding the target catalyst for organic synthesis A21·HBr.

### [Preparation of IRA98·HI]

In a reaction vessel, 2.21 g of a dried product of an anion exchange resin having tertiary amino groups (AmberLite IRA98 (manufactured by Organo Corporation), resin-N(CH₃)₂), 30 mL of water, and 2.28 g of 55% to 58% hydroiodic acid were mixed together, and the mixture was reacted under stirring at room temperature (25° C.) for one hour. Subsequently, the reaction product was filtered, and the filtered residue was washed with water and then dried under reduced pressure, yielding the target catalyst for organic synthesis IRA98·HI.

The synthesis reaction for 4-methyl-1,3-dioxolane-2-one in Example 1-1 to Example 1-3 was conducted using the procedure described below.

In a reaction vessel, 2.5 g of water and a prescribed amount of the obtained catalyst for organic synthesis were added to and mixed with 0.290 g of propylene oxide. A balloon filled with carbon dioxide was then fitted to the vessel, and a reaction was conducted by stirring the mixture under atmospheric pressure at room temperature for 24 hours. Following the reaction, the catalyst for organic synthesis was removed by filtration, yielding crude 4-methyl-1,3-dioxolane-2-one. A ¹H-NMR measurement was conducted using a nuclear magnetic resonance apparatus (Ascend 400 MHz, manufactured by Bruker Corporation), and the reaction conversion rate (%) for 4-methyl-1,3-dioxolane-2-one was determined from the integral value of each spectrum. The results are shown in Table 1.

### Comparative Example 1-1

As a Comparative Example 1-1, the synthesis reaction for 4-methyl-1,3-dioxolane-2-one was conducted in the same manner as the examples, but without using a catalyst. The results are shown in Table 1.

### Comparative Example 1-2

As a Comparative Example 1-2, the synthesis reaction for 4-methyl-1,3-dioxolane-2-one was conducted in the same manner as the examples, but using an I-type catalyst (a strong anion exchange resin having a quaternary ammonium group with an iodide ion counter ion as an ion exchange group) prepared by converting a strong anion exchange resin AmberLite IRA900J CL (manufactured by Organo Corporation) to an iodide form. The results are shown in Table 1.

### (Chemical Structural Formula of Catalyst Used)

resin-N(CH₃)₂·HX
X═I (Comparative Example 1-2)
resin=styrene-divinylbenzene copolymer resin

The synthesis reaction for the catalyst for organic synthesis was conducted using the procedure described below.

### [Preparation of IRA900J·I]

In a reaction vessel were added an anion exchange resin having quaternary amino groups (AmberLite IRA900J CL (manufactured by Organo Corporation), resin-N(CH₃)₃·HCl), and an excess of a 1 N NaOH aqueous solution, and the mixture was stirred at room temperature for 4 hours. The reaction product was then filtered, and the filtered residue was washed with water. Subsequently, 10 mL of the product obtained in the reaction vessel and 1.90 g of 55% to 58% hydroiodic acid were mixed, and the mixture was reacted under stirring at room temperature for 3 hours. The reaction product was then filtered, and the filtered residue was washed with water and dried under reduced pressure, yielding IRA900J·I.

### Comparative Example 1-3

As a Comparative Example 1-3, the synthesis reaction for 4-methyl-1,3-dioxolane-2-one was conducted in the same manner as the examples, but using a dried product of the weak anion exchange resin AmberLyst A21 (manufactured by Organo Corporation).

### Comparative Example 1-4

As a Comparative Example 1-4, the synthesis reaction for 4-methyl-1,3-dioxolane-2-one was conducted in the same manner as the examples, but using a dried product of the strong anion exchange resin AmberLite IRA98 (manufactured by Organo Corporation).

TABLE 1

| | Comparative | Comparative | Comparative | Comparative | | | |
|---|---|---|---|---|---|---|---|
| | Example 1-1 | Example 1-2 | Example 1-3 | Example 1-4 | Example 1-1 | Example 1-2 | Example 1-3 |
| Catalyst | No catalyst added | IRA900J•I type | A21 | IRA98 | A21•HI | IRA98•HI | A21•HBr |
| | — | —N(CH₃)₃•HI | —N(CH₃)₂ | —N(CH₃)₂ | —N(CH₃)₂•HI | —N(CH₃)₂•HI | —N(CH₃)₂•HBr |
| Amount added of catalyst | — | 0.360 g | 0.217 g | 0.220 g | 0.345 g | 0.351 g | 0.297 g |
| Propylene oxide | 85% | 53% | 90% | 91% | 38% | 15% | 22% |
| 4-methyl-1,3-dioxolane-2-one | 0% | 33% | 1% | 1% | 52% | 75% | 52% |
| Propylene glycol (by-product) | 15% | 6% | 10% | 8% | 4% | 3% | 23% |
| Halide (by-product) | 0% | 8% | 0% | 0% | 6% | 7% | 3% |

Based on the test results, it was evident that the weak anion exchange resins having a hydrohalide salt of a tertiary amino group as an ion exchange group from the examples yielded higher reaction conversion rates for 4-methyl-1,3-dioxolane-2-one than the strong anion exchange resin having a quaternary ammonium group and a halide ion or the weak anion exchange resins having a tertiary amino group as an ion exchange group. Further, using ahydroiodide salt (Examples 1-1 and 1-2) produced less by-products than using a hydrobromide salt (Example 1-3).

### Examples 2-1 and 2-2

The synthesis reaction for 4-methyl-1,3-dioxolane-2-one was conducted under various solvent, temperature, and pressure conditions.

The synthesis reaction for 4-methyl-1,3-dioxolane-2-one in Example 2-1 was conducted using the procedure described below.

A prescribed amount of the obtained catalyst for organic synthesis A21·HI was added to and mixed with 0.145 g of propylene oxide in a reaction vessel. A balloon filled with carbon dioxide was then fitted to the vessel, and a reaction was conducted by stirring the mixture under atmospheric pressure at room temperature for 24 hours. Following the reaction, the catalyst for organic synthesis was removed by filtration, yielding crude 4-methyl-1,3-dioxolane-2-one. A ¹H-NMR measurement was conducted using a nuclear magnetic resonance apparatus, and the reaction conversion rate (%) for 4-methyl-1,3-dioxolane-2-one was determined from the integral value of each spectrum. The results are shown in Table 2.

The synthesis reaction for 4-methyl-1,3-dioxolane-2-one in Example 2-2 was conducted using the procedure described below.

A prescribed amount of the obtained catalyst for organic synthesis A21·HI was added to and mixed with 0.145 g of propylene oxide in a pressurized reaction vessel. Carbon dioxide was introduced under pressure at 0.3 MPa, and a reaction was conducted by stirring the mixture at 40° C. for 24 hours. Following the reaction, the catalyst for organic synthesis was removed by filtration, yielding crude 4-methyl-1,3-dioxolane-2-one. A ¹H-NMR measurement was conducted using a nuclear magnetic resonance apparatus, and the reaction conversion rate (%) for 4-methyl-1,3-dioxolane-2-one was determined from the integral value of each spectrum. The results are shown in Table 2.

TABLE 2

| | Example 2-1 | Example 2-2 |
|---|---|---|
| Catalyst | A21•HI | A21•HI |
| | —N(CH₃)₂•HI | —N(CH₃)₂•HI |
| Amount added of catalyst | 0.173 g | 0.173 g |
| Propylene oxide | 84% | 41% |
| 4-methyl-1,3-dioxolane-2-one | 14% | 59% |
| Propylene glycol (by-product) | 1% | 0% |
| Halide (by-product) | 1% | 0% |

Based on the test results, it was evident that the reaction proceeded even in the absence of a solvent, and also evident that the reaction conversion rate could be improved by increasing the temperature and pressure.

### Examples 3-1 to 3-5

In order to investigate the substrate adaptability, the synthesis reactions shown below were conducted to synthesize 4-(chloromethyl)-1,3-dioxolane-2-one and 4-phenyl-1,3-dioxolane-2-one.

### (Reactions Conducted)

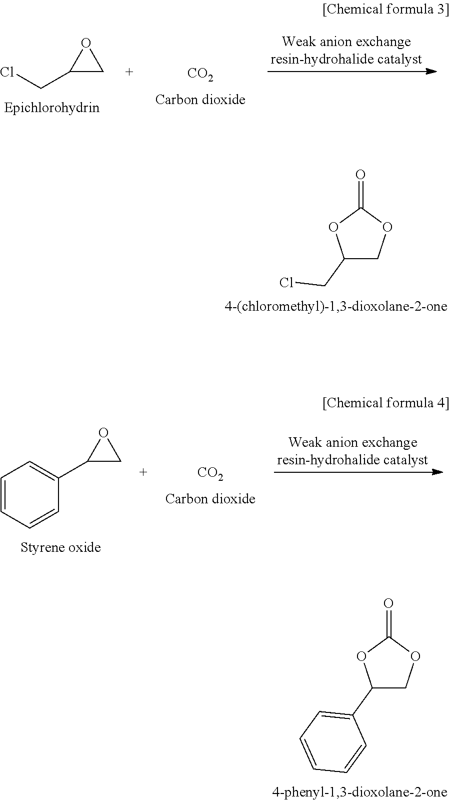 [SMILES: ClCC1CO1 ; "CO2" ; O=C1OCC(CCl)O1 ; c1ccc(C2CO2)cc1 ; "CO2" ; O=C1OCC(c2ccccc2)O1]

### (Chemical Structural Formula of Catalyst Used)

resin-N(CH₃)₂·HX
X═I (Examples 3-2, 3-3 and 3-5), Br (Example 3-1), Cl (Example 3-4)
resin=styrene-divinylbenzene copolymer resin

The synthesis reaction for the catalyst for organic synthesis was conducted using the procedure described below.

### [Preparation of IRA98·HCl]

In a reaction vessel, 2.21 g of a dried product of an anion exchange resin having tertiary amino groups (AmberLite IRA98 (manufactured by Organo Corporation)), 30 mL of water, and 1.04 g of 35% hydrochloric acid were mixed together, and the mixture was reacted under stirring at room temperature for one hour. Subsequently, the reaction product was filtered, and the filtered residue was washed with water and then dried under reduced pressure, yielding the target catalyst for organic synthesis IRA98·HCl.

The synthesis reaction for 4-(chloromethyl)-1,3-dioxolane-2-one in Examples 3-1 and 3-2 was conducted using the procedure described below.

In a reaction vessel, 2.5 g of water and a prescribed amount of the obtained catalyst for organic synthesis were added to and mixed with 0.463 g of epichlorohydrin. A balloon filled with carbon dioxide was then fitted to the vessel, and a reaction was conducted by stirring the mixture under atmospheric pressure at room temperature for 24 hours. Following the reaction, the catalyst for organic synthesis was removed by filtration, yielding crude 4-chloromethyl-1,3-dioxolane-2-one. A ¹H-NMR measurement was conducted using a nuclear magnetic resonance apparatus, and the reaction conversion rate (%) for 4-chloromethyl-1,3-dioxolane-2-one was determined from the integral value of each spectrum. The results are shown in Table 3.

The synthesis reaction for 4-(chloromethyl)-1,3-dioxolane-2-one in Examples 3-3 and 3-4 was conducted using the procedure described below.

A prescribed amount of the obtained catalyst for organic synthesis was added to and mixed with 1.85 g of epichlorohydrin in a pressurized reaction vessel. Carbon dioxide was introduced under pressure at 0.3 MPa, and a reaction was conducted by stirring the mixture at 50° C. for 24 hours. Following the reaction, the catalyst for organic synthesis was removed by filtration, yielding crude 4-(chloromethyl)-1,3-dioxolane-2-one. A ¹H-NMR measurement was conducted using a nuclear magnetic resonance apparatus, and the reaction conversion rate (%) for 4-(chloromethyl)-1,3-dioxolane-2-one was determined from the integral value of each spectrum. The results are shown in Table 3.

The synthesis reaction for 4-phenyl-1,3-dioxolane-2-one in Example 3-5 was conducted using the procedure described below.

A prescribed amount of the obtained catalyst for organic synthesis was added to and mixed with 2.40 g of styrene oxide in a pressurized reaction vessel. Carbon dioxide was introduced under pressure at 0.3 MPa, and a reaction was conducted by stirring the mixture at 50° C. for 24 hours. Following the reaction, the catalyst for organic synthesis was removed by filtration, yielding crude styrene oxide carbonate. A ¹H-NMR measurement was conducted using a nuclear magnetic resonance apparatus, and the reaction conversion rate (%) for 4-phenyl-1,3-dioxolane-2-one was determined from the integral value of each spectrum. The results are shown in Table 3.

TABLE 3

| | Example 3-1 | Example 3-2 | Example 3-3 | Example 3-4 | Example 3-5 |
|---|---|---|---|---|---|
| Catalyst | A21•HBr | A21•HI | IRA98•HI | IR98•HCl | IRA98•HI |
| | —N(CH₃)₂•HBr | —N(CH₃)₂•HI | —N(CH₃)₂•HI | —N(CH₃)₂•HCl | —N(CH₃)₂•HI |
| Amount added of | 0.297 g | 0.345 g | 0.175 g | 0.132 g | 0.175 g |
| catalyst | | | | | |
| Epichlorohydrin | 54% | 30% | 64% | 77% | — |
| 4-(chloromethyl)- | 46% | 70% | 36% | 23% | — |
| 1,3-dioxolane-2-one | | | | | |
| Styrene oxide | — | — | — | — | 92% |
| 4-phenyl-1,3- | — | — | — | — | 8% |
| dioxolane-2-one | | | | | |

Based on the test results, it was evident that cyclic carbonates could be synthesized even when the substrate had a halogenated alkyl group or aromatic group as a substituent.

In this manner, by using the catalysts from the examples, organic compounds such as cyclic carbonates could be synthesized efficiently with superior catalytic activity, and the catalyst could then be separated easily from the reaction system by solid-liquid separation.

1-6. (canceled)

7. A catalyst for organic synthesis,
wherein the catalyst comprises an anion exchange resin having a hydrohalide salt of a tertiary amino group as an ion exchange group, and
wherein the hydrohalide salt is a hydrobromide salt or a hydroiodide salt.

8. The catalyst for organic synthesis according to claim 7, wherein
the catalyst is used for synthesis of a cyclic carbonate.

9. A method for producing an organic compound,
wherein the method comprises using, as a catalyst, an anion exchange resin having a hydrohalide salt of a tertiary amino group as an ion exchange group, and
wherein the hydrohalide salt is a hydrobromide salt or a hydroiodide salt.

10. The method for producing an organic compound according to claim 9, wherein
the organic compound is a cyclic carbonate.
